(19) United States
(12) Patent Application Publication
(10) Pub. No.: US 2003/0161815 A1
(43) Pub. Date: Aug. 28, 2003

(54) Cell delivery system

(75) Inventors: Richard Gregory Wolowacz, Glossop (GB); Michael David Leek, Rixton (GB); Paul David Kemp, Stockport (GB); Jeffrey Keller Teumer, Brookline, MA (US)

Correspondence Address: EDWARDS & ANGELL, LLP, P.O. BOX 9169, BOSTON, MA, 02209, US

(73) Assignee: Intercytex Limited

(21) Appl. No.: 10/074,221

(22) Filed: Feb. 12, 2002

(51) Int. Cl. A61K 7/06

(52) U.S. Cl. 424/93.7; 435/373; 424/70.1

(57) ABSTRACT

The present invention provides a method for inducing organ or tissue formation by delivering inductive cells into an organ or tissue regenerative cellular environment using a controlled delivery device. For the regeneration of hair, the method includes inducing hair follicle formation by delivering inductive cells into a dermal layer using a controlled delivery device. The present invention further provides a method for using a controlled delivery device for the delivery of inductive cells into an organ or tissue regenerative cellular environment to induce organ or tissue formation. The method for using the controlled delivery device includes delivering inductive cells into a dermal layer to induce hair follicle formation.

The present invention relates to delivery of inductive Cells for generation of new organs, for example hair follicles.

Mammalian skin is composed of two layers, an outer layer called the epidermis and an inner layer called the dermis. The epidermis is several cell layers thick, is comprised of mainly keratinocyte cells, and has an external layer of dead cells that are constantly shed from the surface and replaced from below by a basal layer of cells, the stratum germinativum. The dermis comprises a network of collagenous extracellular material, elastic fibres, blood vessels, nerves and hair follicles with associated sebaceous glands.

During embryogenesis, the establishment of a dermal papilla is vital to the development of hair follicles and associated modified structures like sebaceous glands. The dermal papilla is a group of specialised dermal fibroblast cells, derived from the embryonic mesoderm. These dermal papilla cells begin to aggregate in the dermis just below the epidermis. Above the dermal papilla an epidermal plug, or peg, of cells develops and proliferates growing into the dermis towards the dermal papilla. The mesoderm-derived dermal papilla and the ectoderm-derived epidermal plug communicate via molecular signals with the result of further proliferation of epidermal matrix cells and differentiation into the various sheath and hair fibre structures. Thus the development of a hair follicle requires a continuum through induction, initiation, elongation and differentiation stages.

A mature hair follicle comprises a bulb containing the dermal papilla cells, a hair shaft extending from the bulb through to the exterior of the epidermis, and a dermal sheath which provides an external covering of tissue around the bulb and along the length of the follicle. The hair follicle extends down through the dermis, a hypodermis (a loose layer of connective tissue below the dermis), and a fat or adipose layer. In adults, molecular signals between the dermal papilla and the epidermal component of a follicle cause the hair to enter an active (anagen) growth phase from an inactive (telogen) phase.

Baldness (known medically as alopecia) is defined as the absence of hair from an area of the body, especially where hair normally exists. Baldness can exist or arise for several reasons. Lack of hair can be caused by the non-presence of hair follicles, for example for genetic reasons. Hair loss can be caused by destruction (for example scarring), disease, infection and/or disruption of the natural hair growth cycle (for example, due to insensitivity to hormones).

Several methods for treating baldness have been attempted. One approach has been to use pharmaceutical drugs (such as Minoxidil [RTM; Rogaine, Upjohn] and Finasteride [RTM; Propecia, Merck]). However, pharmaceuticals have achieved limited success in restoring natural hair growth.

Another approach, particularly for hair loss, has been hair transplantation, for example where tissue comprising hair follicles is transplanted from a site where the hair follicles are insensitive to dihydroxytestosterone (for example the back of the head) to a sensitive site where hair has been lost. This autograft approach has some problems including “doll-like hair”.

In other work, chimaeric hair has been generated by grafting tissue containing inductive dermal papilla or dermal sheath cells from a donor into the epidermis of a non-donor recipient (see for example WO0132840). Such chimaeric hair tends to grow in variable directions and the method does not result in natural-looking hair.

Attempts have been made to inject donor cells into a recipient using a conventional hypodermic needle and syringe. The method does not allow cells to be delivered reproducibly or in controlled amounts into a subcutaneous compartment at an appropriate depth from the surface of the epidermis. Hair follicles induced with the bulb too close to the epidermal/dermal junction are susceptible to being pulled out when placed under a mechanical stress such as combing or brushing. Reproducibly obtaining the correct angle of hair shaft growth has also not been possible.

The prior art methods of treating baldness are therefore not optimal. The methods are restricted by the inability to control factors such as the density, orientation and positioning of induced or transplanted hair follicles. Furthermore, mechanical techniques (grafting, transplantation and injection) tend to be painful.

In the field of drug delivery, controlled delivery devices have provided safer, more reliable and more effective delivery of fluid drugs than the conventional hypodermic needle and syringe. Examples of such improvements are one or more high velocity driven needles (see WO0009184), a high pressure fluid delivery system (see U.S. Pat. No. 5,540,657 and U.S. Pat. No. 6,224,567), a tracked injection needle (see U.S. Pat. No. 5,620,421), or needleless delivery means (see US20010027293 A1).

The present inventors have established that, unexpectedly, controlled delivery devices can be utilised to deliver appropriate cells into a recipient to provide effective treatments. In particular, controlled delivery devices are found to be useful for treating baldness.

According to the present invention there is provided a method for inducing hair follicle formation, comprising delivering inductive dermal sheath cells and/or inductive dermal papilla cells into a dermal layer using a controlled delivery device. In the prior art, controlled delivery devices were developed to improve delivery of fluid drugs, and their use or effectiveness in delivering living cells for induction of tissues or organs such as hair follicles has not been taught or suggested. The present method is advantageous in that a physician or other practitioner will be able to repeatedly, accurately and precisely deliver cells in a cell suspension or bolus into a subcutaneous compartment. The method also allows viable cells to be delivered in a reproducible volume of cell suspension while minimizing the amount of pain experienced by the patient. The method thus provides a significant improvement over prior art methods of treating baldness, in particular over using a traditional hypodermic needle and syringe and over other mechanical techniques such as grafting.

In one embodiment, cells are allowed to aggregate, or are induced to aggregate, into clumps of cells of the appropriate volume and cell number before being injected subcutaneously. Cells or aggregates of cells may be placed in a formulation, such as hyaluronic acid or glycosaminoglycans, that includes a substance (or substances) which increases the viscosity of the injected material in order to protect the cells during handling and injection. Cells or aggregates of cells may be placed in a formulation, for example one including fibronectin and/or collagen, that enhances the microenvironment of cells after implantation, in order to facilitate cell migration or cell-cell interaction.

The inductive dermal sheath cells and/or inductive dermal papilla cells may be derived from a variety of sources. One source is mesenchymal stem cells derived from bone marrow (available from Osiris, for example). Another source is bone marrow mesodermal progenitor cells (see WO 01/11011—Catharine Verfaillie's multipotent adult progenitor cells). Yet a further source is hematopoietic stem cells derived from human bone marrow. Another source of cells are pluripotent cells derived from the skin (Toma, J. G. el al., 2001, Nature Cell Biol. 3: 778-784; Aegera Therapeutices Inc. & Curis Inc [both US]). Alternatively, the inductive dermal sheath cells and/or inductive dermal papilla cells may be derived from embryonic stem cells. Another source is embryonic carcinoma cells which have been suitably differentiated towards a DP phenotype for hair using known methods. (Teratomas from which embryonic carcinoma cell lines can be derived have hair and teeth-like structures: embryonic carcinoma cells are commercially available from Layton Biosciences [US], for example.) A further source is reprogrammed cells, for example, autologous cells such fibroblasts which have been “reprogrammed” by dermal papilla cells or embryonic carcinoma cells to induce hair formation (for reprogramming of cells, see WO0049138).

Cells with a desired functionality of hair inducibility may be stably maintained in culture using known methods (see for example: U.S. Pat. No. 5,851,831, for long tern subculture of dermal papilla cells; and the methods disclosed in WO01/74164).

The inductive dermal sheath cells and/or inductive dermal papilla cells may be delivered to a depth from an outer surface where normal hair follicles form in vitro (eg. in cultured skin) or in vivo (ie. in a person or other mammal). For example, the depth may be 0.5-4.0 mm into human tissue. The controlled delivery device allows the depth of delivery to be precisely determined and consistently reproduced, but is also adjustable for a particular delivery situation. Delivery of the inductive cells to the correct depth allows induced hair follicles to be imbedded in the dermis so that developed hairs will be better anchored and less susceptible to mechanical stresses such as pulling, combing or brushing.

The inductive dermal sheath cells and/or inductive dermal papilla cells may be delivered at a given angle within the dermal layer.

A growing hair follicle will not necessarily automatically orientate itself properly. According to the invention the inductive dermal sheath cells and/or inductive dermal papilla cells may be delivered in a track (or channel) formed by the controlled delivery device and oriented towards an outer surface. The track may be contiguous with the host epidermis. A track provides a pathway which allows a nascent hair follicle to grow in the correct direction towards the surface of the skin and connect with the surface epidermis surrounding the track. In addition, the angle of the track can be varied, allowing the nascent hair follicle to grow at an appropriate angle relative to the outer surface. This achieves a good cosmetic result because hair follicles grow at different angles in different regions of the scalp and a more robust hair follicle. In one embodiment, the controlled delivery device used to generate the track has a needle with blunt end and an orifice on the lateral side near the tip (for example, an orifice approximately 0.5 mm from the tip), allowing cells to be implanted along the needle track.

The inductive dermal sheath cells and/or inductive dermal papilla cells may be from a source autologous or allogeneic to the dermal layer. Cells from an allogeneic source should preferably be screened for viruses before use.

Preferably, the controlled delivery device comprises one or more high velocity driven needles (for example as claimed in WO0009184), a high pressure fluid delivery system (for example as claimed in U.S. Pat. No. 5,540,657 or U.S. Pat. No. 6,224,567), a tracked injection needle (for example as claimed in U.S. Pat. No. 5,620,421) or is needleless (for example as claimed in US20010027293 A1).

Further provided according to the present invention is a method as described herein additionally including one or more steps resulting in the development of a mature hair follicle.

Also provided according to the present invention is the use of a controlled delivery device for the delivery of inductive dermal sheath cells and/or inductive dermal papilla cells into a dermal layer to induce hair follicle formation. The features pertaining to the method elaborated herein are also applicable for this use.

It will also be appreciated that the invention described herein may be more generally applicable so as to provide a method for inducing organ or tissue formation. The method may comprise delivering inductive cells into an organ or tissue regenerative cellular environment using a controlled delivery device. Furthermore, the invention covers the use of a controlled delivery device for the delivery of inductive cells into a regenerative cellular environment to induce organ or tissue formation. Using a controlled delivery device also allows for cells to be delivered to a specific location for therapeutic purposes.

Experimental

A full-thickness piece of human male scalp skin is placed into a small amount of medium. Using fine forceps and scalpel under stereomicroscopic observation, intact hair follicles are individually dissected from the piece of skin. Each individual follicle is further dissected, first to remove excess connective tissue, then to remove the lower dermal sheath and dermal papilla from the epidermal portion of the follicle. Once the sheaths and papillae are separated from the rest of the follicle, the dermal papillae are separated from the sheaths at the stalk that connects the two structures using a hypodermic needle as a scalpel. Dermal papillae are placed into culture medium and allowed to attach. Typically, 2-3 papilla are placed into a well of a 12-well culture dish. After several days, papillae are analysed to detect cells growing out from them. When explants have been grown for a further10-12 days, cultures are trypsinised and replated at a density of 2-3×10³ cells/cm². After one week, cells are confluent and ready for transplantation. Medium used for propagation of cells is Chang Medium (Irvine Scientific, Santa Ana, Calif.) combined with human keratinocyte conditioned medium.

To prepare cells for transplantation, cells are removed from culture dishes by trypsinisation. A high concentration cell suspension is prepared (10³- 10⁴ cells per μl) and then loaded into an Imprint cell delivery device (see WO0009184). The needle of the device has a blunt end and an orifice that is on the lateral side approximately 0.5 mm from the tip, allowing cells to be implanted along the needle track. This allows the implanted cells to be in close proximity to the epidermis and it also facilitates cell migration along the track. The angle of the track determines the angle of hair growth, and the depth ensures that the hair follicle will be well anchored in the dermis.

Full-thickness human female skin from a hairless region of the body (obtained from breast reduction surgery) is grafted on athymic mice anesthetised with ketamine/xylazine. Using the delivery device, the hair inductive cells are implanted into the grafted skin. The depth of the needle tip is 0.2-0.8 mm below the surface of the skin and depends upon the depth setting of the device and the angle at which the needle is inserted. For each separate injection, a cell suspension volume of 1-5 μl is injected, delivering 2-10×10³ hair inductive cells. After the injections, mice are allowed to recover from the anesthesia and are housed individually in separate cages. After approximately 6 weeks, the mice are monitored for hair induction at the injection sites.

In general terms, the skilled person should appreciate that the efficiency of the novel organ formation may be dependent on the following factors:

the number of inductive cells injected at a given site;

the exact position and distance of inductive cells with respect to the dermal-epidermal junction (for example the depth of the needle track);

the inductive status of the inductive cells;

the passage number of the inductive cells;

the culture media used in culture for expanding the inductive cells;

how the inductive cells are “aggregated” prior to loading the delivery system (nucleation of aggregates using a number of beads [e.g. microcarrier beads] may be useful); and

the production of a suitable wound in the dermis.

These factors can be modified to obtain optimal results for a given application.

1. A method for inducing hair follicle formation, comprising delivering inductive dermal sheath cells and/or inductive dermal papilla cells into a dermal layer using a controlled delivery device.

2. A method for inducing hair follicle formation in a dermal layer lying beneath an outer skin surface, comprising delivering inductive dermal sheath cells and/or inductive dermal papilla cells into the dermal layer using a controlled delivery device.

3. The method according to either one of claim 1 or claim 2, wherein the inductive dermal sheath cells and/or inductive dermal papilla cells are derived from mesenchymal stem cells and/or mesodermal progenitor cells and/or hematopoietic stem cells and/or embryonic stem cells and/or embryonic carcinoma cells and/or reprogammed cells.

4. The method according to any one of the preceding claims, wherein the inductive dermal sheath cells and/or inductive dermal papilla cells are delivered to a depth from an outer skin surface, the depth corresponding to a position where normal hair follicles form in vitro or in vivo.

5. The method according to claim 4, wherein the depth is 0.5-4.0 mm into human tissue.

6. The method according to any one of the preceding claims, wherein the inductive dermal sheath cells and/or inductive dermal papilla cells are delivered at a given angle within the dermal layer.

7. The method according to any one of the preceding claims, wherein the inductive dermal sheath cells and/or inductive dermal papilla cells are from a source autologous or allogeneic to the dermal layer.

8. The method according to any one of the preceding claims, wherein the controlled delivery device comprises one or more high velocity driven needles (for example as claimed in WO0009184), a high pressure fluid delivery system (for example as claimed in U.S. Pat. No. 5,540,657 or U.S. Pat. No. 6,224,567), a tracked injection needle (for example as claimed in U.S. Pat. No. 5,620,421) or is needleless (for example as claimed in US20010027293 A1).

9. The method according to any one of the preceding claims which additionally includes one or more steps resulting in the development of a mature hair follicle.

10. Use of a controlled delivery device for the delivery of inductive dermal sheath cells and/or inductive dermal papilla cells into a dermal layer to induce hair follicle formation.

11. The use according to claim 10, wherein the inductive dermal sheath cells and/or inductive dermal papilla cells are derived from mesenchymal stem cells and/or mesodermal progenitor cells and/or hematopoietic stem cells and/or embryonic stem cells and/or embryonic carcinoma cells and/or reprogrammed cells.

12. The use according to either one of claim 10 or claim 11, wherein the inductive dermal sheath cells and/or inductive dermal papilla cells are delivered to a depth an outer skin surface, the depth corresponding to a position where normal hair follicles form in vitro or in vivo.

13. The use according to claim 12, wherein the given depth is 0.5-4.0 mm into human tissue.

14. The use according to any one of claims 10-13, wherein the inductive dermal sheath cells and/or inductive dermal papilla cells are delivered at a given angle within the dermal layer.

15. The use according to any one of claims 10-14, wherein the inductive dermal sheath cells and/or inductive dermal papilla cells are from a source autologous or allogeneic to each other and/or the dermal layer.

16. The use according to any one of claims 10-15, wherein the controlled delivery device comprises one or more high velocity driven needles (for example as claimed in WO0009184), a high pressure fluid delivery system (for example as claimed in U.S. Pat. No. 5,540,657 or U.S. Pat. No. 6,224,567), a tracked injection needle (for example as claimed in U.S. Pat. No. 5,620,421) or is needleless (for example as claimed in US20010027293 A1).

17. The use of a controlled delivery device for the delivery of inductive cells into a regenerative cellular environment to induce organ or tissue formation.

18. A method for inducing organ or tissue formation, comprising delivering inductive cells into an organ or tissue regenerative cellular environment using a controlled delivery device.

19. The method according to claim 18, additionally comprising one or more steps resulting in the development of a functional organ or tissue.
